(19) United States
(12) Patent Application Publication
(10) Pub. No.: US 2026/0335143 A1
(43) Pub. Date: Oct. 29, 2026

(54) DRESSING HAVING AN INTEGRAL CLOSURE DEVICE

(71) Applicant: Solventum Intellectual Properties Company, Maplewood, MN (US)

(72) Inventors: Jonathan G. REHBEIN, San Antonio, TX (US); Christopher J. CARROLL, San Antonio, TX (US); Shervin JAHANIAN, San Antonio, TX (US); Luke PERKINS, San Antonio, TX (US)

(21) Appl. No.: 19/111,627

(22) PCT Filed: Sep. 7, 2023
(86) PCT No.: PCT/IB2023/058879

§ 371 Date: Mar. 13, 2025

(51) Int. Cl. A61F 13/0203; A61B 17/00; A61B 17/08; A61F 13/00; A61F 13/02

(52) U.S. Cl. CPC A61F 13/0226; A61B 17/085; A61F 13/0289; A61B 2017/00867; A61F 2013/00936

(57) ABSTRACT

An apparatus for generating a force at a tissue site is described. The apparatus can include a closure device formed from a super-elastic material. The closure device can have a first side, a second side, a perimeter portion, and a center portion disposed inboard of the perimeter portion, the center portion configured to exert the force at the tissue site. The apparatus can also include an adhesive layer disposed adjacent to the first side of the closure device, and a top cap layer disposed adjacent and coupled to the second side of the closure device.

### CROSS-REFERENCE TO RELATED APPLICATIONS

This application is a U.S. National Stage Entry of PCT International Application No. PCT/IB2023/058879, filed on Sep. 7, 2023, which claims the benefit of priority to U.S. Provisional Application No. 63/408,165, filed on Sep. 20, 2022, which is incorporated herein by reference in its entirety.

### TECHNICAL FIELD

The invention set forth in the appended claims relates generally to tissue treatment systems and more particularly, but without limitation, to a dressing having an integral closure device.

### BACKGROUND

Whether the etiology of a tissue site, or damaged area of tissue, is trauma, surgery, or another cause, proper care of the tissue site is important to the outcome. Currently, an opening on the epidermis may be closed using sutures, staples, clips, and other mechanical devices to allow the skin to be held and pulled. Such devices often cause puncture wounds or other wounds. Moreover, if severe edema occurs, tremendous pressure may be placed on the closure device, and the pressure may cause harm to the tissue site.

Where a tissue site is an area without an opening in the epidermis, proper care can also improve a rate of healing of the tissue site. For example, application of an appropriate force, such as compression, apposition, or distention force can help control blood flow to the tissue site to reduce swelling or encourage growth factors and re-building of tissue. Current devices used to apply these forces may require cumbersome systems and/or rely on materials that may be unable to apply a consistent force to the area over time.

While the clinical benefits of closure of an opening through the epidermis and the application of an appropriate force to a tissue site are widely known, improvements to therapy systems, components, and processes may benefit healthcare providers and patients.

### BRIEF SUMMARY

New and useful systems, apparatuses, and methods for closing a tissue site are set forth in the appended claims. Illustrative embodiments are also provided to enable a person skilled in the art to make and use the claimed subject matter.

For example, in some embodiments, an apparatus for generating a force at a tissue site is described. The apparatus can include a closure device formed from a super-elastic material. The closure device can have a first side, a second side, a perimeter portion, and a center portion disposed inboard of the perimeter portion, the center portion configured to exert the force at the tissue site. The apparatus can also include an adhesive layer disposed adjacent to the first side of the closure device, and a top cap layer disposed adjacent and coupled to the second side of the closure device.

More generally, a dressing for covering a tissue site is described. The dressing can include a mesh having a center portion and a perimeter portion. The center portion can be configured to generate distension in the tissue site. The dressing can also include an adhesive layer disposed adjacent to the mesh, and a film layer disposed adjacent to the mesh, the adhesive layer adhering the film layer to the mesh.

Alternatively, other example embodiments may describe a method of manufacturing a dressing. A nitinol mesh can be provided. The nitinol mesh can be constrained in a mold. The mold can have a convex portion and a concave mating portion. The nitinol mesh can be heated, and the nitinol mesh can be removed from the mold. A double-sided adhesive can be laminated to the nitinol mesh, and the nitinol mesh can be encapsulated with a top cover.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a perspective view of an example embodiment of a plurality of apparatuses disposed at a tissue site in accordance with some embodiments;

FIG. 2 is a sectional view of a cover taken along line 2-2 of FIG. 1 and illustrating additional features that may be associated with some embodiments;

FIG. 3 is a sectional view of another example of the cover taken along line 2-2 of FIG. 1 and illustrating additional features that may be associated with some embodiments;

FIG. 4 is a schematic view of the cover having a closure device during the process of applying the cover in accordance with some embodiments;

FIG. 5 is a schematic view of the cover having the closure device during the process of applying the cover in accordance with some embodiments;

FIG. 6 is a schematic view of the cover having the closure device during the process of applying the cover in accordance with some embodiments;

FIG. 7 is a sectional view of another cover having another closure device that may be associated with some embodiments;

FIG. 8 is a perspective view illustrating the cover of FIG. 7 disposed at the tissue site;

FIG. 9 is a sectional view of the cover of FIG. 7 taken along line 9-9 of FIG. 8;

FIG. 10 is a plan view of the tissue site having a plurality of closure devices disposed across an incision of the tissue site in accordance with some embodiments; and

FIG. 11 is a plan view of the tissue site after a plurality of the closure devices disposed at the tissue site.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, a tissue site may be a target site for growth of additional tissue that may be harvested and transplanted.

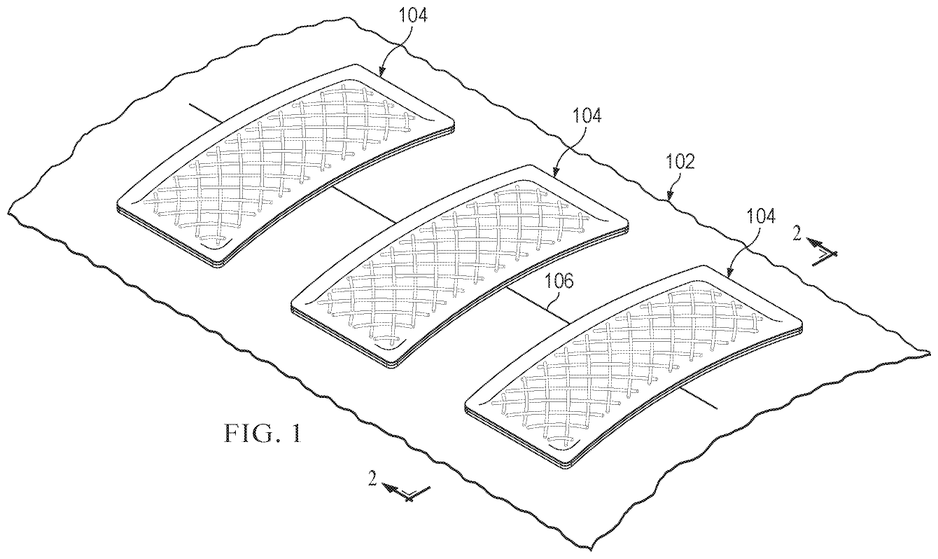

FIG. 1 is a perspective view of an example embodiment of a plurality of apparatuses having an integral closure device disposed at a tissue site 102. Each apparatus can provide a force at a tissue site in accordance with this specification. In some embodiments, each apparatus may comprise a cover 104.

In some embodiments, the cover 104 may provide a bacterial barrier and protection from physical trauma. The cover 104 may also be constructed from a material that can reduce evaporative losses and provide a fluid seal between two components or two environments, such as between a therapeutic environment and a local external environment. The cover 104 may comprise or consist of, for example, an elastomeric film or membrane that can provide a seal adequate to maintain a negative pressure at a tissue site for a given negative-pressure source. The cover 104 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38 °C and 10% relative humidity (RH). In some embodiments, an MVTR up to 5,000 grams per square meter per twenty-four hours may provide effective breathability and mechanical properties. In some example embodiments, the cover 104 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. An attachment device may be used to attach the cover 104 to an attachment surface, such as undamaged epidermis, a gasket, or another cover. The attachment device may take many forms.

In operation, the cover 104 may be placed within, over, on, or otherwise proximate to the tissue site 102. If the tissue site 102 includes an incision 106 or other opening in tissue, the cover 104 may be placed over or across the incision. In other embodiments, the cover 104 may overlay the entire incision 106. In some embodiments, each cover 104 may overlay a portion of the incision 106. For example, more than one cover may be disposed along a length of the incision 106. The cover 104 may be sealed to an attachment surface near the incision 106. For example, the cover 104 may be sealed to undamaged epidermis peripheral to the incision 106. Application of the cover 104 to the tissue site 102 may cause the cover 104 to exert a force on the tissue site 102. In some embodiments, the cover 104 may draw edges of the incision 106 toward each other, urging the incision 106 to close.

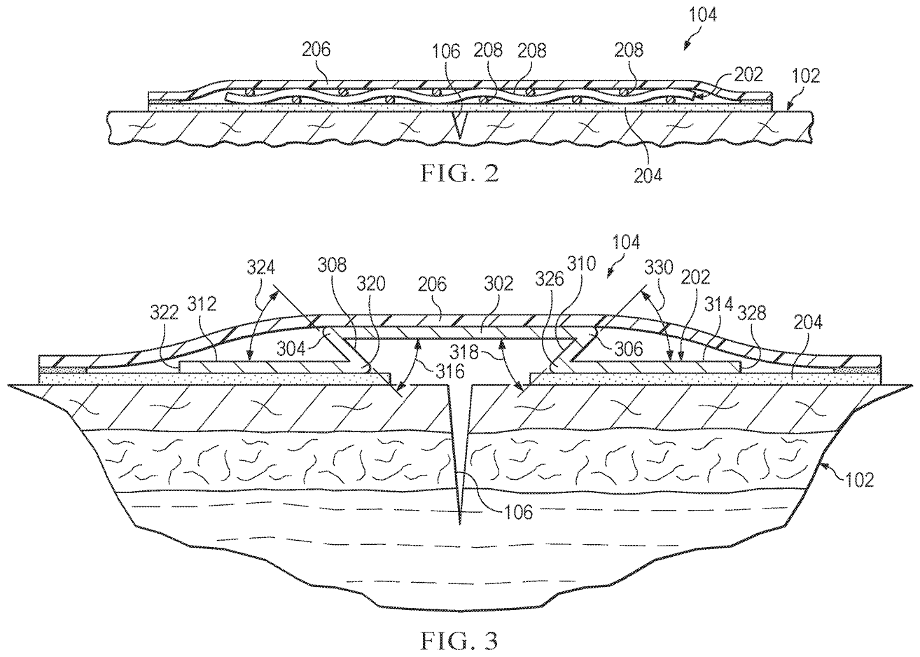

FIG. 2 is a sectional view of the cover 104 taken along line 2-2 of FIG. 1 and illustrating additional features that may be associated with some embodiments. In some embodiments, the cover 104 can include a closure device 202, an adhesive layer 204, and a top cap layer 206. The closure device 202 can be disposed between the adhesive layer 204 and the top cap layer 206. In some embodiments, the top cap layer 206 and the adhesive layer 204 can encapsulate or surround the closure device 202.

In some embodiments, the closure device 202 can be a mesh. The mesh can comprise a plurality of strands 208 formed from threads, fibers, or wires. In some embodiments, the mesh of the closure device 202 can be formed by interweaving the plurality of strands 208. For example, a first plurality of strands 208 may be positioned perpendicular to a second plurality of strands 208. In other embodiments, the strands 208 may be positioned at non-perpendicular angles to each other. The strands 208 may be woven to form a mesh or net. In other embodiments, the plurality of strands 208 can overlay each other without being woven. In some embodiments, the strands 208 may have a pitch between about 6 mm and about 25 mm. The strands 208 may have an average effective diameter between about 0.1 millimeters (mm) and about 1 mm. In some embodiments, the closure device 202 can be formed from a super-elastic material. For example, the strands 208 can be formed from a super-elastic material. Preferably, the super-elastic material is biocompatible, non-ferromagnetic, and MR-conditional. In some embodiments, the closure device 202 can generate compressive, appositional, and/or distention forces. In some embodiments, the closure device 202 can be tuned. The closure device 202 may have two variables that can be tuned. First, the final shape of the closure device 202 in its austenite state can be tuned. And second, the temperature at which the closure device 202 returns to its austenite state can be tuned. The transformation temperature can be tuned slightly via variations in composition. For example, a ratio of nickel versus titanium or the presence of other trace metals in the alloy can change the austenite state. The temperature can be tuned even further via heat treatment processes. The variables used in heat treating the closure device 202 include duration and temperature of the heat treatment processes. Preferably, the transformation temperature would be equal or less than body heat (37 °C) or at room temperature (20 °C). In some embodiments, the closure device 202 may retain elasticity over time.

In some embodiments, the super-elastic material may be a nickel-titanium alloy, such as nitinol. Nitinol may have properties related to its martensite-austenite transformation and a selectable transition temperature allowing the alloy to have shape-memory properties in response to a particular range of temperatures. An alloy having shape-memory properties can be deformed when cold but return to its pre-deformed shape in response to heat. In some embodiments, the nitinol can be heat treated to set a transition temperature for the nitinol. In some embodiments, the closure device 202 can have a martensite-austenite transition temperature lower than about 20 degrees Celsius, permitting the closure device 202 to exhibit shape-memory properties at room temperatures. In other embodiments, the closure device 202 can have a martensite austenite transition temperature at about 34 degrees Celsius, permitting the closure device 202 to exhibit shape-memory properties in response to body heat. In other embodiments, the closure device 202 can have a martensite austenite transition temperature at about 100 degrees Celsius, permitting the closure device 202 to exhibit shape-memory properties in response to an external heat source.

Nitinol can have a large elastic range and can deform around eight times more than ordinary spring steel while still returning to its original shape. Nitinol may have an elastic modulus for austenite nitinol between about 75 gigapascals (GPa) and about 83 GPa. Nitinol may have an elastic strain for austenite nitinol of about 10%. In contrast, steel may have an elastic modulus between about 193 GPa and about 210 GPa and an elastic strain of about 1%. Nitinol can have a relatively flat force or stress over a wide range of deformation or strain, allowing for a known force to be applied across an incision, regardless of how much the user pre-stressed the dressing. In some embodiments, the closure device 202 may exert a force between about 0 kilopascals and about 13.8 kilopascals. In other embodiments, the closure device 202 may exert larger forces selected for the particular therapy of the tissue site.

In some embodiments, the adhesive layer 204 can be an attachment device. An attachment device may be a medically-acceptable, pressure-sensitive adhesive configured to bond the cover 104 to epidermis at the tissue site 102. In some embodiments, for example, some or all of the cover 104 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel. as previously described. In some embodiments, the adhesive layer 204 can comprise a high-tack adhesive. In some embodiments, the adhesive layer 204 can comprise a skin friendly adhesive. In some embodiments, the adhesive layer 204 may be a single-sided adhesive. In some embodiments, the adhesive layer 204 can comprise a 3 M 2484 GSA or a Tegaderm acrylic adhesive. For example, the adhesive layer 204 can have an adhesion to low density polyethylene at 180 degrees of about 430 g/25 mm width or 2.8N/25.4 mm.

In some embodiments, the top cap layer 206 may be a film layer. For example, the top cap layer 206 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the top cap layer 206 may comprise INSPIRE 2301 having an MVTR (upright cup technique) of 2600 g/m 2/24 hours and a thickness of about 30 microns.

In other embodiments, the top cap layer 206 may also be an adhesive or an attachment device. For example, the top cap layer 206 may be a medically-acceptable, pressure-sensitive adhesive configured to bond the cover 104 to epidermis around a tissue site. In some embodiments, for example, some or all of the cover 104 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel. as previously described. In some embodiments, the top cap layer 206 may be a high-tack adhesive. In some embodiments, the top cap layer 206 can comprise a skin friendly adhesive. In some embodiments, the top cap layer 206 may be a single-sided adhesive. In some embodiments, the top cap layer 206 can comprise a 3 M 2484 GSA or a Tegaderm acrylic adhesive.

The closure device 202 can be laminated between the top cap layer 206 and the adhesive layer 204. For example, the top cap layer 206 may have an adhesive surface configured to be positioned adjacent to the closure device 202. The adhesive surface of the top cap layer 206 may couple to the closure device 202, securing the closure device 202 to the top cap layer 206. The adhesive layer 204 can be positioned adjacent to the closure device 202 opposite the top cap layer 206. The adhesive layer 204 may be coupled to the closure device 202 and the top cap layer 206, encapsulating or surrounding the closure device 202 between the adhesive layer 204 and the top cap layer 206. In some embodiments, the adhesive layer 205 may be a double-sided adhesive. The adherent surface adjacent to the closure device 202 may couple the adhesive layer 204 to the closure device 202 and the top cap layer 206. In other embodiments, the adhesive layer 204 may be a single-sided adhesive. The surface adjacent to the closure device 202 may be non-adherent, and the adherent surface of the top cap layer 206 may also adhere to the adhesive layer 204 through the closure device 202. In still other embodiments, the top cap layer 206 may be welded to the adhesive layer 204 about a perimeter of the top cap layer 206 and the adhesive layer 204.

In operation the cover 104 can be stretched across the tissue site 102, such as an incision. As the cover 104 is stretched across the tissue site 102, the closure device 202 may be deformed. For example, the closure device 202 may be deformed or stretched in a direction perpendicular to the incision 106 of the tissue site 102. After the cover 104 is secured to the tissue site 102 by the adhesive layer 204, the force deforming the closure device 202 may be released, and the super-elastic properties of the closure device 202, such as the nitinol mesh, can help pull the incision 106 of the tissue site 102 closed.

FIG. 3 is a sectional view of another example of the cover 104 taken along line 2-2 of FIG. 1 and illustrating additional features that may be associated with some embodiments. In some embodiments, the closure device 202 can comprise a spring or a nitinol spring. The closure device 202 can be laminated between the top cap layer 206 and the adhesive layer 204. For example, the top cap layer 206 may be a polyurethane film configured to be positioned adjacent to the closure device 202. The adhesive layer 204 can be positioned adjacent to the closure device 2020 opposite the top cap layer 206. The adhesive layer 204 may be coupled to the closure device 202 and the top cap layer 206, encapsulating or surrounding the closure device 202 between the adhesive layer 204 and the top cap layer 206. In some embodiments, the adhesive layer 205 may be a double-sided adhesive. The adherent surface adjacent to the closure device 202 may couple the adhesive layer 204 to the closure device 202 and the top cap layer 206.

In some embodiments, the closure device 202 can comprise a beam 302 having a first end 304 and a second end 306. A first arm 308 can be coupled to the first end 304, and a second arm 310 can be coupled to the second end 306. The first arm 308 may be a rod having a first end and a second end opposite the first end. The first arm 308 may have a shaft extending between the first end and the second end. In some embodiments, the first end of the first arm 308 may be coupled to the first end 304 of the beam 302. The first arm 308 may extend from the beam 302. In some embodiments, the second end of the first arm 308 may be closer to a centerline of the beam 302 than the first end. For example, the first arm 308 may form an angle 316 with the beam 302 having the first end 304 as a vertex of the angle 316. The angle 316 may be less than about 90 degrees. In some embodiments, the angle 316 may be between about 30 degrees and about 60 degrees, and preferably about 45 degrees.

The second arm 310 may be a rod having a first end and a second end opposite the first end. The second arm 310 may have a shaft extending between the first end and the second end. In some embodiments, the first end of the second arm 310 may be coupled to the second end 306 of the beam 302. The second arm 310 may extend from the beam 302. In some embodiments, the second end of the second arm 310 may be closer to a centerline of the beam 302 than the first end. For example, the second arm 310 may form an angle 318 with the beam 302 having the second end 306 as a vertex of the angle 318. The angle 318 may be less than about 90 degrees. In some embodiments, the angle 318 may be between about 30 degrees and about 60 degrees, and preferably about 45 degrees.

The closure device 202 may further include a first anchor 312 and a second anchor 314. The first anchor 312 may be a beam or rod having a first end 320 and a second end 322 opposite the first end 320. The first anchor 312 may have a shaft extending between the first end 320 and the second end 322. The first end 320 of the first anchor 312 may be coupled to the second end of the first arm 308. The first anchor 312 may extend from the first arm 308 away from the beam 302. In some embodiments, the first anchor 312 may be parallel to the beam 302. In some embodiments, the second end 322 of the first anchor 312 may be further from the centerline of the beam 302 than the first end 320. For example, the first anchor 312 may form an angle 324 with the first arm 308 having the first end 320 as a vertex of the angle 324. The angle 324 may be less than about 90 degrees. In some embodiments, the angle 324 may be between about 30 degrees and about 60 degrees, and preferably about 45 degrees.

The second anchor 314 may be a beam or rod having a first end 326 and a second end 328 opposite the first end 326. The second anchor 314 may have a shaft extending between the first end 326 and the second end 328. The first end 326 of the second anchor 314 may be coupled to the second end of the second arm 310. The second anchor 314 may extend from the second arm 310 away from the beam 302. In some embodiments, the second anchor 314 may be parallel to the beam 302. In some embodiments, the second end 328 of the second anchor 314 may be further from the centerline of the beam 302 than the first end 326. For example, the second anchor 314 may form an angle 330 with the second arm 310 having the first end 326 as a vertex of the angle 330. The angle 330 may be less than about 90 degrees. In some embodiments, the angle 330 may be between about 30 degrees and about 60 degrees, and preferably about 45 degrees.

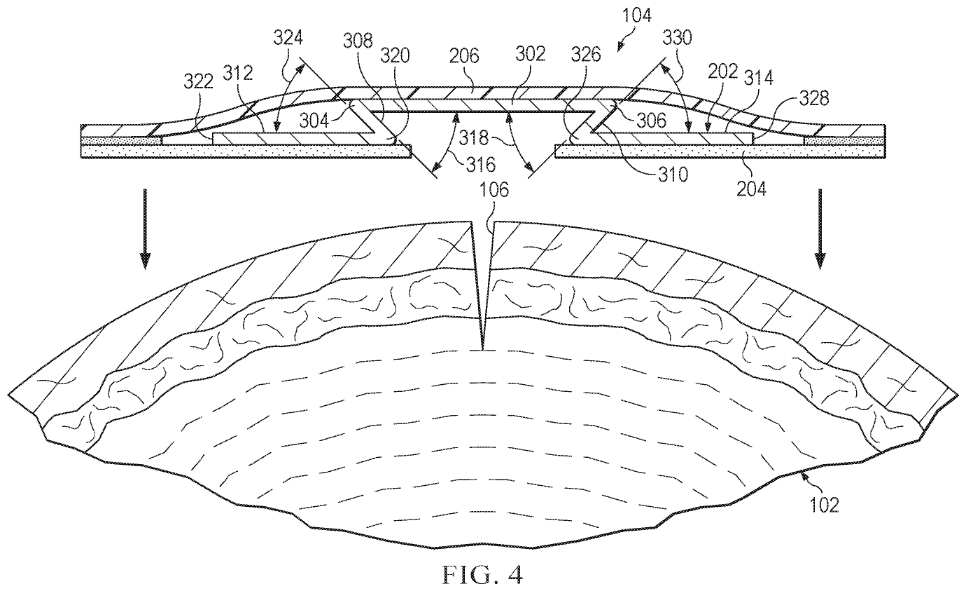

FIG. 4 is a schematic view of the cover 104 having the closure device 202 during the process of applying the cover 104 in accordance with some embodiments. The cover 104 can be stretched across the tissue site 102, such as the incision 106 of the tissue site 102. As the cover 104 is stretched across the tissue site 102, the closure device 202 may be deformed. For example, the first anchor 312 and the second anchor 314 may be pulled in opposite directions. Pulling the first anchor 312 and the second anchor 314 in opposite directions can cause the angle 316, the angle 318, the angle 324, and the angle 330 to increase as the first end 320 and the first end 326 move away from each other.

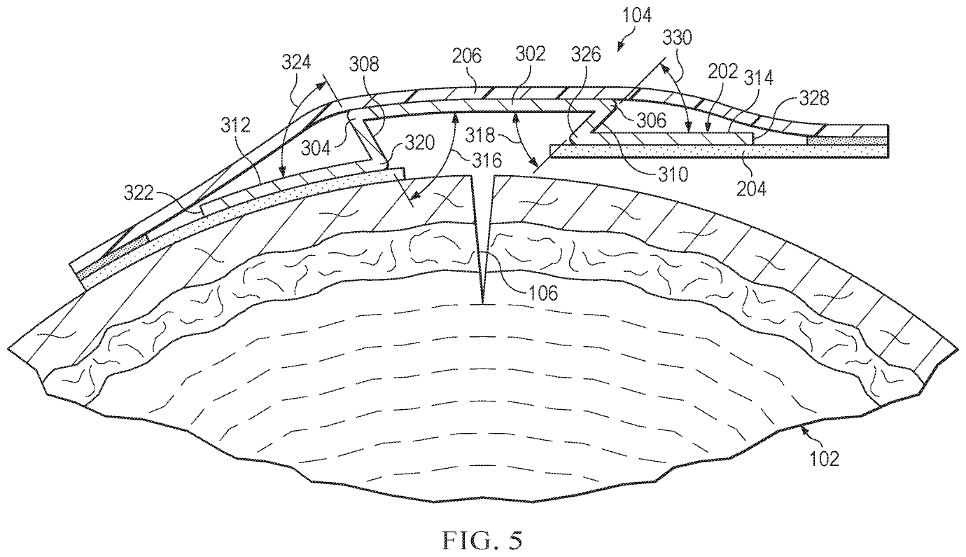

FIG. 5 is a schematic view of the cover 104 having the closure device 202 during the process of applying the cover 104 in accordance with some embodiments. In some embodiments, the first anchor 312 may be secured on one side of the incision 106 by the adhesive layer 204. After the first anchor 312 of the cover 104 is secured to the tissue site 102 on one side of the incision 106, the closure device 202 may be further deformed. For example, the second anchor 314 may be moved further away from the first anchor 312. The second anchor 314 may be secured to the tissue site 102 on an opposite side of the incision 106 from the first anchor 312. For example, the adhesive layer 204 may secure the second anchor 314 to the tissue site 102.

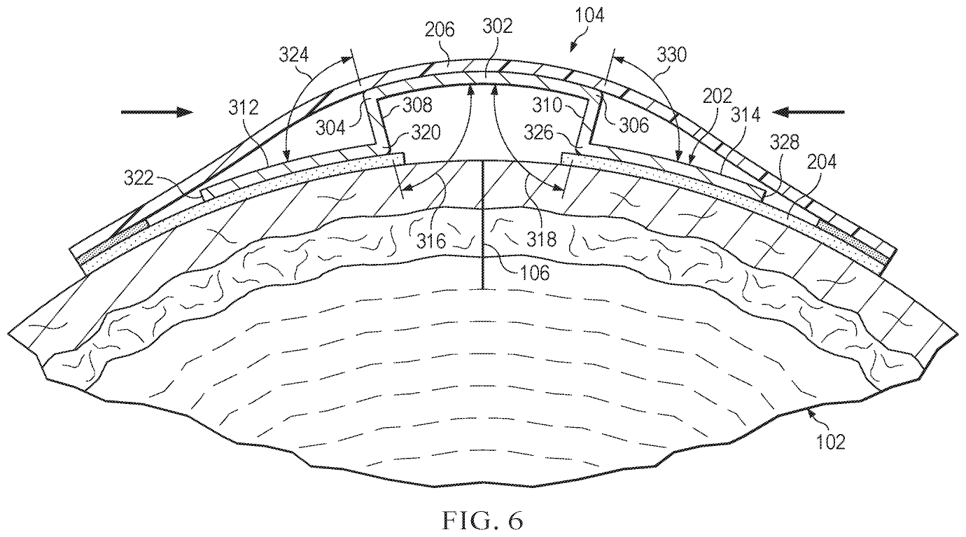

FIG. 6 is a schematic view of the cover 104 having the closure device 202 during the process of applying the cover 104 in accordance with some embodiments. After the first anchor 312 and the second anchor 314 are secured on opposite sides of the incision 106 of the tissue site 102, the force deforming the closure device 202 may be released. The closure device 202 can be urged to the original shape of the closure device 202. For example, if the closure device 202 is formed from nitinol, the super-elastic properties of the nitinol can cause the first end 326 and the first end 320 to move toward each other, urging the angle 316, the angle 318, the angle 324, and the angle 330 toward their original measurement. The force of the nitinol urging the angle 316, the angle 318, the angle 324, and the angle 330 toward their original measurement can exert a force pushing edges of the incision 106 of the tissue site 102 toward each other to close the incision 106 of the tissue site 102.

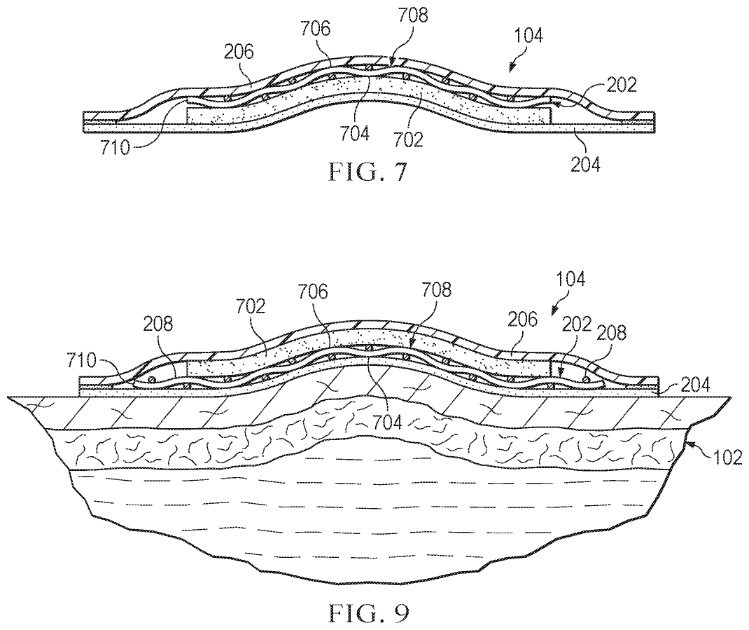

FIG. 7 is a sectional view of another cover 104 having another closure device 202 that may be associated with some embodiments. As shown in FIG. 7, the cover 104 may include a fluid storage device 702. In some embodiments, the fluid storage device 702 may be disposed between the closure device 202 and the adhesive layer 204. In other embodiments, the fluid storage device 702 may be disposed between the closure device 202 and the top cap layer 206.

In some embodiments, the closure device 202 may comprise a mesh having a pre-formed shape. For example, the closure device 202 may have a center portion 708 and a perimeter portion 710. The perimeter portion 710 may surrounding the center portion 708. In some embodiments, the center portion 708 may have a concave side 704 and a convex side 706. The concave side 704 may be disposed proximate to the adhesive layer 204, and the convex side may be disposed proximate to the top cap layer 206. In some embodiments, the concave side 704 may have a radius of curvature between about 0 mm and about 10 mm. In some embodiments, the perimeter portion 710 may be substantially flat and have a width from the union with the center portion between about 2 centimeters (cm) and about 3 cm.

In some embodiments, the concave side 704 and the convex side 706 can be formed by constraining the mesh of the closure device 202 in a mold. The mold can have the desired shape for the concave side 704 and the convex side 706. After constraining the mesh, the mesh can be heat treated. For example, the mesh can be heated in a salt bath or a furnace to between about 500 degrees Celsius and about 550 degrees Celsius. The mesh can retain the shape of the mold following the heat treatment process giving the closure device 202 the concave side 704 and the convex side 706.

The fluid storage device 702 may be an absorbent that stores, or immobilizes, the liquid from a tissue site. The absorbent may be any substance capable of storing a liquid, such as exudate. For example, the absorbent may form a chemical bond with exudate from the tissue site. Non-limiting examples of the absorbent include super absorbent fiber/particulates, hydrofibre, sodium carboxymethyl cellulose, and/or alginates. In some exemplary embodiments, the absorbent may be formed of a superabsorbent polymer (SAP). Generally, relative to their mass, SAPs can absorb and retain large quantities of liquid, and in particular water. SAPs may be used to hold and stabilize or solidify wound fluids. The SAPs used to form the absorbent may be of the type often referred to as “hydrogels,” “super-absorbents,” or “hydrocolloids.” When disposed within a dressing, the SAPs may be formed into fibers or spheres to manifold reduced pressure until the SAPs become saturated. Spaces or voids between the fibers or spheres may allow a reduced pressure that is applied to a dressing to be transferred within and through the absorbent. In some embodiments, fibers of the absorbent may be either woven or non-woven. In some embodiments, the absorbent may comprise a substrate in which the SAPs may be dispersed as pellets throughout and/or embedded as a sheet-like layer within the substrate.

The SAPs may be formed in several ways, for example, by gel polymerization, solution polymerization, or suspension polymerization. Gel polymerization may involve blending of acrylic acid, water, cross-linking agents, and ultraviolet (UV) initiator chemicals. The blended mixture may be placed into a reactor where the mixture is exposed to UV light to cause crosslinking reactions that form the SAP. The mixture may be dried and shredded before subsequent packaging and/or distribution. Solution polymerization may involve a water-based monomer solution that produces a mass of reactant polymerized gel. The monomer solution may undergo an exothermic reaction that drives the crosslinking of the monomers. Following the crosslinking process, the reactant polymer gel may be chopped, dried, and ground to its final granule size. Suspension polymerization may involve a water-based reactant suspended in a hydrocarbon-based solvent. However, the suspension polymerization process must be tightly controlled and is not often used.

SAPs absorb liquids by bonding with water molecules through hydrogen bonding. Hydrogen bonding involves the interaction of a polar hydrogen atom with an electronegative atom. As a result, SAPs absorb water based on the ability of the hydrogen atoms in each water molecule to bond with the hydrophilic polymers of the SAP having electronegative ionic components. High absorbing SAPs are formed from ionic crosslinked hydrophilic polymers such as acrylics and acrylamides in the form of salts or free acids. Because the SAPs are ionic, they are affected by the soluble ionic components within the solution being absorbed and will, for example, absorb less saline than pure water. The lower absorption rate of saline is caused by the sodium and chloride ions blocking some of the water absorbing sites on the SAPs. If the fluid being absorbed by the SAP is a solution containing dissolved mineral ions, fewer hydrogen atoms of the water molecules in the solution may be free to bond with the SAP. Thus, the ability of an SAP to absorb and retain a fluid may be dependent upon the ionic concentration of the fluid being absorbed. For example, an SAP may absorb and retain de-ionized water up to 500 times the weight of the dry SAP. In volumetric terms, an SAP may absorb fluid volumes as high as 30 to 60 times the dry volume of the SAP. Other fluids having a higher ionic concentration may be absorbed at lower quantities. For example, an SAP may only absorb and retain a solution that is 0.9% salt (NaCl) up to 50 times the weight of the dry SAP. Since wound fluids contain salts, such as sodium, potassium, and calcium, the absorption capacity of the SAP may be reduced if compared to the absorption capacity of deionized water.

In some embodiments, the absorbent may comprise a KERRAMAX CARE™ Super-Absorbent Dressing material available from Kinetic Concepts, Inc. of San Antonio, Texas. For example, the absorbent may comprise a superabsorbent laminate comprised of 304 g.s.m. FAVOR-PAC™ 230 superabsorbent powder glued by PAFRA™ 8667 adhesive between two layers of 50 g.s.m. LIDRO™ non-woven material. In some embodiments, the absorbent may comprise an absorbent available from Gelok International. The presence of the absorbent may also help to minimize fluid loss or reflux.

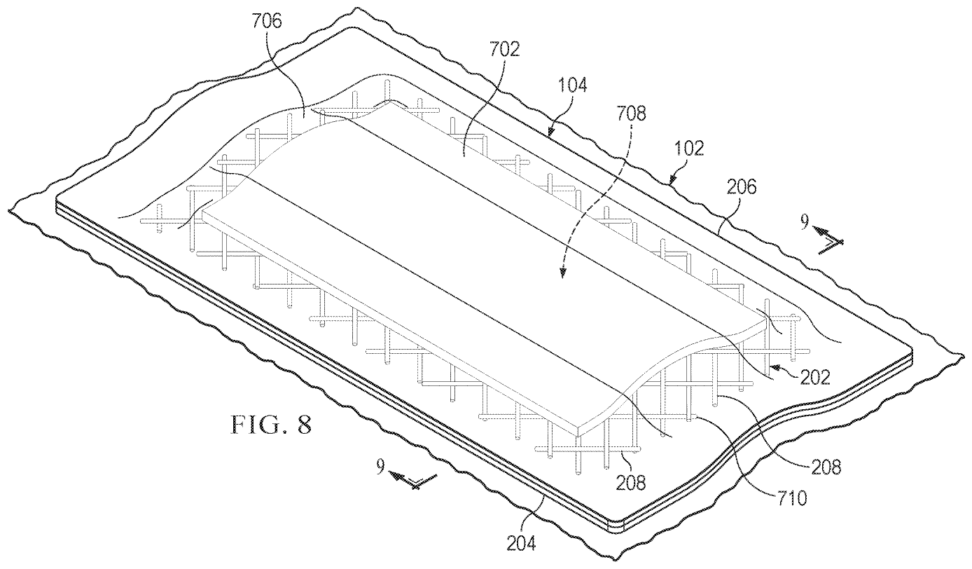

FIG. 8 is a perspective view illustrating the cover 104 of FIG. 7 disposed at the tissue site 102. In some embodiments, the tissue site 102 may be an area to tissue without a penetration of the dermis or epidermis but which may receive beneficial effects from the application of a distension force. In operation, the cover 104 can be stretched across the tissue site 102. As the cover 104 is stretched across the tissue site 102, the closure device 202 may be deformed. For example, the closure device 202 may be deformed or stretched in a direction perpendicular to an area of desired distension of the tissue site 102.

FIG. 9 is a sectional view of the cover 104 of FIG. 7 taken along line 9-9 of FIG. 8. After the cover 104 is secured to the tissue site 102 by the adhesive layer 204, the force deforming the closure device 202 may be released, and the super-elastic properties of the nitinol mesh and the curvature of the concave side 704 and the convex side 706 may create a distension force in an area of tissue at the tissue site 102. In some embodiments, the fluid storage device 702 may receive fluid, such as perspiration and secure it away from the surface of the tissue site 102 to prevent maceration and decrease the rate of hydration of the adhesive layer 204. In some embodiments, the storage of fluid may permit the cover 104 to remain at the tissue site 102 for longer periods of duration. In some embodiments, the distension created by the closure device 202 can distend skin and fascia, dilating lymph and blood vessels and permitting increased blood flow to the tissue site 102.

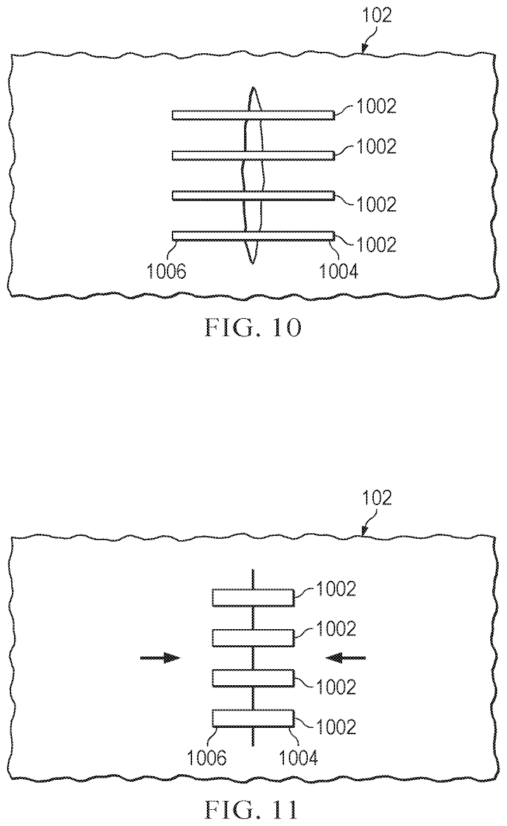

FIG. 10 is a plan view of the tissue site 102 having a plurality of closure devices 1002 disposed across an incision of the tissue site 102 in accordance with some embodiments. In some embodiments, the closure device 1002 can comprise a rod having a first end 1004 and a second end 1006. Both the first end 1004 and the second end 1006 may have a high-tack, adhesive encapsulating the respective end. For example, the adhesive may be a medically-acceptable, pressure-sensitive adhesive configured to bond the cover 104 to epidermis around a tissue site. In some embodiments, for example, some or all of the cover 104 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel. as previously described. In some embodiments, the adhesive can comprise a 3 M 2484 GSA or a Tegaderm acrylic adhesive.

In operation, each closure device 1002 can be stretched across the tissue site 102, such as an incision. For example, the first end 1004 can be coupled to a first side of the incision of the tissue site 102 with the adhesive. The second end 1006 may be stretched across incision of the tissue site 102 generally perpendicular to the incision of the tissue site 102. As the closure device 1002 is stretched across the tissue site 102, the closure device 1002 may be deformed. For example, the closure device 1002 may be deformed or stretched in a direction perpendicular to the incision of the tissue site 102. The second end 1006 of the closure device 1002 may be secured to an opposite side of the incision of the tissue site 102 by the adhesive.

FIG. 11 is a plan view of the tissue site 102 after a plurality of closure devices 1002 have been disposed at the tissue site 102. After the second end 1006 is secured to the tissue site 102 by the adhesive, the force deforming the closure device 1002 may be released, and the super-elastic properties of the nitinol may help pull the incision of the tissue site 102 closed.

The systems, apparatuses, and methods described herein may provide significant advantages. For example, the closure devices described herein provide for primary dermal closure of an incision. The devices described herein can provide compressive, appositional, and/or distention forces. The closure devices can be programmable to provide a desired force that limits tensioning of an incision as is the case with traditional sutures or staples. The closure devices described herein may not lose elasticity over time as is the case with other closure devices relying on films or elastics alone. Furthermore, the devices described herein provide a relatively flat force or stress over a wide range of deformation or strain. The closure devices can also provide a known force regardless of how much the closure device is pre-stressed.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for generating a force at a tissue site, the apparatus comprising:
a closure device formed from a super-elastic material, the closure device having a first side, a second side, a perimeter portion, and a center portion disposed inboard of the perimeter portion, the center portion configured to exert the force at the tissue site;
an adhesive layer disposed adjacent to the first side of the closure device; and
a top cap layer disposed adjacent and coupled to the second side of the closure device.

2. The apparatus of claim 1, wherein the closure device comprises a spring.

3. The apparatus of claim 2, wherein the closure device further comprises:
a beam having ends;
a pair of arms, each arm having a first end coupled to a respective end of the beam, each arm coupled to the beam at an acute angle; and
a pair of anchors, each arm having a second end coupled to a respective end of an anchor at an acute angle.

4. The apparatus of claim 1, wherein the closure device comprises a strip configured to generate a compressive force parallel to an axis of the strip.

5. The apparatus of claim 1, wherein the closure device comprises a mesh.

6. The apparatus of claim 1, wherein the first side of the center portion has a concave shape.

7. The apparatus of claim 6, wherein the second side of the center portion has a convex shape.

8. The apparatus of claim 6, wherein the force is a distention force.

9. The apparatus of claim 1, wherein:
the adhesive layer comprises an adherent side and a non-adherent side opposite the adherent side;
the top cap layer comprises an adhesive having an adherent side and a non-adherent side opposite the adherent side; and
the non-adherent side of the adhesive layer being positioned adjacent to the closure device and the adherent side of the top cap layer being positioned adjacent to the closure device, the adherent side of the top cap layer adhering the top cap layer to the closure device and the adhesive layer.

10. The apparatus of claim 1, further comprising an absorbent disposed between the closure device and the top cap layer.

11. The apparatus of claim 10, further comprising an opening through the adhesive layer, the opening configured to permit fluid flow across the adhesive layer into the absorbent.

12. The apparatus of claim 1, wherein the super-elastic material comprises nitinol.

13. The apparatus of claim 1, wherein the top cap layer comprises a polyurethane film.

14. A dressing for covering a tissue site, the dressing comprising:
a mesh having a center portion and a perimeter portion, the center portion configured to generate distension in the tissue site;
an adhesive layer disposed adjacent to the mesh; and
a film layer disposed adjacent to the mesh, the adhesive layer adhering the film layer to the mesh.

15. The dressing of claim 14, wherein the center portion of the mesh comprises a concave shape adjacent to the adhesive layer and a convex shape adjacent to the film layer.

16. The dressing of claim 14, wherein the mesh comprises nitinol.

17. The dressing of claim 14, further comprising a fluid retention device disposed between the mesh and the film layer.

18. A method of manufacturing a dressing, the method comprising:
providing a nitinol mesh;
constraining the nitinol mesh in a mold, the mold having a convex portion and a concave mating portion;
heating the nitinol mesh;
removing the nitinol mesh from the mold;
laminating a double-sided adhesive to the nitinol mesh; and
encapsulating the nitinol mesh with a top cover.

19. The method of claim 18, wherein heating the nitinol mesh comprises heating the nitinol mesh to a temperature in a range of about 500 to about 550 degrees Celsius.

20. The method of claim 18, wherein the method further comprises laminating an absorbent layer between the top cover and the double-sided adhesive.

21. (canceled)
